(19) United States
(12) Patent Application Publication
(10) Pub. No.: US 2015/0320539 A1
(43) Pub. Date: Nov. 12, 2015

(54) PACKAGING AND TRANSFER SYSTEM FOR AN IMPLANT APPLICATION

(71) Applicant: QVANTEQ AG, Zürich (CH)

(72) Inventors: Arik ZUCKER, Zürich (CH); Stefano BUZZI, Birmensdorf (CH); Armin W. MÄDER, Richterswil (CH)

(21) Appl. No.: 14/410,335

(22) PCT Filed: May 31, 2013
(86) PCT No.: PCT/EP2013/061247

§ 371 Date: Dec. 22, 2014

(30) Foreign Application Priority Data
Jun. 28, 2012 (CH) 00910/12

(51) Int. Cl. A61F 2/00; A61M 25/00; B65D 53/02; A61F 2/95; A61F 2/97

(52) U.S. Cl. CPC A61F 2/0095; A61F 2/95; A61F 2/97; B65D 53/02; A61M 25/002; A61F 2002/9522

(57) ABSTRACT

A packaging system comprises having packaging for an implant (6) and/or a catheter (7), which has a receptacle (1) for the implant (6) and/or the catheter (7) which receptacle has at its distal end a closure (2) and an opening or actuating mechanism with a closable and expandable axial passage (5). An insertion aid for the insertion of a removal device and/or a wire (14) into an inner lumen of the implant and/or catheter can be provided by a sleeve-like insertion element (10, 100, 101, 102, 103, 104), which is arranged at least partially within the axial passage (5) in the closure (2), or is formed by the closing element. An opposite, distal end forms an insertion opening (120) for the removal device and/or the wire (14) into the insertion element. The packaging can include at a rear end a transport device (7) for moving the implant, or the device for use of the implant, relative to the packaging through the closure. A coupling device (15) with a passage (17) which device moves relative to a receptacle in the packaging can connect to the closure, which passage can be connected to an inlet port (12) of a container or an insertion system (11).

The present invention relates to a packaging for an implant or a device for use of an implant and a transfer system for removal of the implant or the device for use of an implant out of the packaging and for insertion of the implant into an insertion system, in particular for a transfer of a stent mounted on an insertion catheter out of an inert packaging via a guide wire through a hemostasis valve, or also called Y-connector, into a lock, which forms an access to the patient.

In many areas of medicine, implants are used for treatment of various diseases or defects. For example, in the field of angioplasty, vascular prosthetic devices in the form of stents are used for vasodilation in the treatment of lesions. A stent generally has a multiplicity of crosspieces which together form a tubular form. The stent length extends between a proximal and a distal end and the stent lumen as passage with a compressible diameter. In the dilated or respectively released state the stent takes on an expanded diameter e.g. for support of a blood vessel. Essentially a distinction is made between balloon-expanding or self-expanding stents. Self-expanding stents are mounted on a tube catheter. Balloon-expanding stents are put on a non-expanded balloon before their implantation, which balloon is located at the tip of an insertion catheter.

The stents implanted in blood vessels however entail certain risks for the patient. Among other things restenosis or thrombosis can form on the structures of the stent. It is therefore common to treat the surface of stents; e.g. stents are provided with an antithrombogenic and hemocompatible surface. In so doing the stents are subjected among other things to a specific cleaning procedure. The stents are provided in clean or respectively purified state in an at least sterile packaging, which also serves as storage container, and are removed therefrom only shortly before the insertion of the stent into a body lumen. The stents are preferably stored in an inert environment in order to be able to ensure a high-purity surface.

For preparation of the stent for an implantation, it is taken out of the packaging and is put in an insertion system. The stent can thereby be put already on an insertion catheter. In a conventional procedure the insertion catheter is taken manually from its packaging simultaneously with the mounted stent, e.g. on the support wire located in the guide wire lumen of the insertion catheter. For installation of the insertion catheter on a guide wire, which has been positioned beforehand in the patient through a lock in a body lumen to be treated, as a rule the support wire is removed from the insertion catheter and the insertion catheter, starting from the tip, is threaded on the guide wire. As a rule this takes place by hand. The stent mounted on the insertion catheter is thereby inevitably contaminated with blood, with lubricant from the insertion catheter as well as with small and minute particles from the environment, among other things from the gloves or the like used by the treatment personnel. The stent mounted on the insertion catheter and thus located close to the catheter tip is sometimes taken hold of directly by the treatment personnel with the gloves because this simplifies considerably the threading of the guide wire.

Known from US 2003/187493 is a system in which a stent is accommodated in a protective sheath and is protected against contamination. The protective sheath however has to be removed before insertion of a guide wire in a dilation catheter of the stent. During the insertion of the guide wire in the insertion catheter and the insertion of the insertion catheter through a hemostasis valve e.g. in the form of a Y-connector the stent is completely exposed to a contaminating atmosphere and manual grasp.

It is an object of the present invention to create a packaging and a transfer system for removal of an implant out of a packaging and for insertion of the implant into an insertion system in which the implant is protected in a reliable way from contamination and manual access, which facilitates the insertion of an implant into an insertion system and thus into a body lumen and simplifies the handling of the implant.

Furthermore it is an object of the present invention to make simple and quick the insertion of a wire, such as e.g. a guide wire of a catheter, into a catheter or respectively into a stent, thereby protecting the stent from contamination and/or manual grasp, and to provide a system in which the stent can remain in a packaging during the insertion of the wire.

This object is achieved by the invention through a packaging and a transfer system for implants according to claims 1 and 12. Furthermore the object is achieved through a packaging system and a use of a sleeve-like element according to claims 15 and 29. Advantageous embodiments and further embodiment examples are described in the dependent claims.

A packaging for an implant or a device for use of an implant according to the present invention has at a front, i.e. a distal, end a closure with an opening mechanism and at a rear, i.e. proximal, end a transport device for moving of the implant relative to the packaging, i.e. to a receptacle or to a container of the packaging, through the closure. Adjoining the closure is a coupling device with a passage which is designed to be coupled to an inlet port of a container or of an insertion system. The packaging forms with the container or the insertion system and the coupling device a transfer system for transfer of the implant or a device for use of an implant out of the packaging into the container or the insertion system for insertion of the implant into a body lumen.

The implant or respectively the device for use of an implant can be brought from the packaging via the insertion system into a body lumen without being touched and without being exposed to a contaminating environment. Thus the risk for a patient of complications during ingrowth of the implant into body tissue is thereby reduced. The packaging preferably forms an inert container for the implant or the device for use of the implant. The container can be mounted, for example, on a blister and can be shrink-wrapped in a sterile bag.

The packaging and the transfer system according to the invention can be used advantageously during the implantation of stents. In so doing the stent is disposed on the transport device. The transport device serves the purpose of making possible movement of the stent relative to the packaging, or respectively to the receptacle of the packaging, i.e. making possible transport out of the receptacle by pushing or pulling. An insertion catheter is preferably used for this purpose. However a wire, a tube, a stick or the like can also be used. With the insertion catheter the stent can be pushed through the closure on the distal end of the packaging, which has a passage in an opened state, and through the passage of the coupling device into the inlet port, such as e.g. a hemostasis valve, of the insertion system.

When bringing the stent into a body lumen usually the stent or respectively the insertion catheter is attached via a guide wire of the insertion system, so that the stent is able to be pushed along the guide wire to the body lumen. For threading of the stent or respectively of the transport device, e.g. in the form of a catheter, an insertion aid can be provided, according to the invention, on the packaging and/or on the coupling device as guide for the guide wire. The insertion aid guides the guide wire to the distal opening of the insertion catheter at the catheter tip inside the packaging so that it can be pushed into the insertion catheter. Thus the stent or the insertion catheter does not have to be positioned manually on the guide wire. The stent can be transferred out of the packaging to the body lumen in a contact-free way.

Provided according to the invention is thus a packaging system with a packaging for an implant, in particular in the form of a stent, and/or a device for use of the stent in the form of a catheter with an insertion aid for insertion of a removal device and/or a wire, such as, for example, a guide or support wire, into the implant and/or into the utilization device, or respectively into the catheter, i.e. into an inner lumen of the implant and/or of the catheter. The packaging comprises a receptacle for the implant and/or the utilization device, or respectively the catheter, which receptacle has at its distal end a closure and an opening mechanism with a closable and expandable axial passage with which the axial passage can be radially constricted or expanded. The closure has a closure element which can be elastically deformable, for example. The insertion aid is provided by means of a sleeve-like insertion element which is disposed at least partially inside the axial passage of the closure or is formed by the closure, e.g. is designed in one piece with the closure element. A distal opening of the insertion element forms an insertion opening for the wire or the removal device in the insertion element.

The sleeve-like insertion element can be formed e.g. by a piece of tube of polymer, Teflon® (PTFE) or also of a ferrous alloy. In one embodiment the insertion element is so rigid that it is not compressed by the closure mechanism. A proximal opening of the insertion element can thereby come out into the receptacle or into the passage. If the proximal opening comes out into the receptacle it can enclose a tip of the catheter or of the implant in the packaging. In this way the catheter is sealed. If the proximal opening comes out in the closure element, the closure element can be compressed in the area without insertion element by the closure mechanism so that the receptacle of the packaging is sealed. The closure element is thereby elastic in such a way that a wire can be pushed through also in compressed state of the closure element.

According to another embodiment of the invention, the insertion element can be designed to be elastic at least at a proximal end and can be used as insertion aid for insertion of the wire or of the removal device in an inner lumen of the implant and/or of the catheter. The elastic end is positioned in an adjacent way at an open catheter end, i.e. at the distal catheter tip, and is compressed in such a way that it is adjacent to the catheter end in a flush way. Preferably the elastic end encloses the catheter end completely. The wire, or respectively the removal device is inserted at the opposite distal end into the sleeve-like element and is pushed through the elastically designed end into the catheter. During the pushing through the elastic end can expand elastically, if necessary.

The packaging according to the invention, the packaging system and the transfer system can be used with a device for use of the implant, such as e.g. a balloon of a balloon catheter. The balloon can thereby be stored in the packaging alone, i.e. without a stent mounted thereon. Also other kinds of catheters can serve as device for use of the implant, which catheters are generally used with diseases of arteriosclerosis. These can be catheters e.g. which are used for imaging methods, such as, for example, intravascular ultrasound methods (IVUS) or an optical coherence tomography (OCT), or aspiration catheters for removal of thrombosis, etc. The utilization device can also be used for catheters with other applications. The invention will be explained in the following, by way of example, for an implant, in particular a stent. The same application is however also suitable for a device for use of an implant.

The coupling device can be provided as a separate coupling piece which is coupled to the packaging and also to the insertion system, such as e.g. to the hemostasis valve, or can be connected to a container. Alternatively the coupling device can be provided in a fixed way on the packaging. For example, the coupling device can be designed as one piece with the packaging or also just firmly connected, e.g. through adhesion, thereto. The coupling device can have a snap device, screw device, a frictionally engaged connection and/or a bayonet coupling for coupling to the packaging and/or to a container or an insertion system.

In one embodiment the packaging has an elongated, in particular cylindrical, receptacle with a front and a rear opening. Provided on the front and rear opening is a closure, e.g. in the form of a cap, for closing of the openings. At least the front, i.e. the distal, closure has preferably an axial passage expandable in radial direction, which passage can be opened and closed by means of the opening mechanism. The cap as closure can be part of the opening mechanism or can form the opening mechanism. The cap can e.g. be rotatable relative to the receptacle or displaceable around the passage for opening or for closing.

The packaging can be constructed in a way similar to the packaging as it is explained in FIGS. 3 and 4 and the related description of the parallel application of the applicant “Anordnung and Verfahren zur Bereitstellung eines Stents zur Implantation mit Umhüllung” (CH 00048/12).

The closure can thus have a closure element in the form of an elastically deformable seal with the axial passage which e.g. by means of a rotation element, such as the cap, is deformable in such a way that the passage opens or closes upon rotation of the rotation element. In a variant the cap can be disposed with a screw connection over the opening of the receptacle so that it is movable axially with respect to the receptacle. The seal is conical at its end inserted in the receptacle. The tapered surface abuts a conical surface inside the receptacle. Through a screwing movement of the cap the tapered surface of the seal is pushed axially and is pressed against the conical surface. The closure element is thereby compressed radially, so that the passage is pressed together. Owing to the elasticity of the closure element the passage opens as soon as the cap is again unscrewed. In principle it is also possible to push the cap manually in axial direction in order to open or to close the passage of the closure. In this embodiment the cap, the closure element, or respectively the seal, and the conical surface of the receptacle form together the opening mechanism. The opening mechanism serves at the same time as closure mechanism for the axial passage in that this passage is radially constricted. The opening and closure mechanism thus form an actuation mechanism for opening and closing of the axial passage at the distal end of the packaging.

The implant can be stored in a tube element that extends through the receptacle of the packaging and runs through the passage of the closure. The tube element is compressed in the area of the closure and thereby closed as described in the previously mentioned parallel application of the applicant.

An elastically deformable seal with an axial passage can likewise be provided on the rear end of the packaging through and into which the transport device runs from outside the packaging. The passage can be compressible in a way similar to the closure at the front end of the packaging and can thereby firmly clamp the transport device, such as e.g. the insertion catheter. The passage can be opened for insertion of the implant for preparation of an implantation.

Other fixing devices for the transport device are in principle conceivable. The tube element for storage of the implant can also extend through the rear passage and can be closed by this rear passage.

According to the invention the coupling device can be provided by means of an insertion aid, in particular by means of the sleeve-like insertion element. The coupling device can also be designed as an elongated coupling piece which has at its opposite ends in each case a coupling mechanism for coupling to the packaging and if applicable to the container or the insertion system, such as e.g. the hemostasis valve. The coupling piece can thereby be firmly coupled, e.g. on the cap of the packaging. Thus the coupling piece can be used in coupled state in order to operate the opening mechanism. The coupling piece can however remain movable relative to the packaging or respectively the receptacle also in coupled state so that the opening mechanism can be operated independently of a movement of the coupling piece. Alternatively the coupling mechanism can be disposed e.g. directly on the cap. The passage of the coupling device runs substantially centrally through the coupling device and is thus closed in circumferential direction so that a manual grasping, for example of the implant, is prevented while the implant is locked by the coupling piece.

The coupling mechanism can create a releasable or a firm connection. As mentioned, it can be provided by means of a snap device, screw device, a frictionally engaged connection and/or a bayonet coupling. The snap device can be formed by elastically movable snap hooks which snap in e.g. behind an axial stop on the packaging, e.g. on the cap. The screw device can be formed by a threaded connection between coupling piece and packaging, or respectively cap. The frictionally engaged connection can take place, for example, through a simple pressing of the coupling piece into a depression or swelling of the coupling piece, suitable with respect to the shape of the packaging. Further coupling mechanisms are conceivable.

The passage of the coupling device, or respectively of the insertion aid, extends in axial direction and in coupled state connects to the axial passage of the closure. In opened state of the packaging closure there results therefore an exit out of the packaging, which exits runs through the passage of the closure and through the passage of the coupling device or respectively of the insertion aid. The passage of the coupling device comes out directly into the inlet port of the container of the insertion system, or respectively of the hemostasis valve. The implant is thereby protected not only against e.g. manual grasp but also the insertion of the insertion system takes place automatically during insertion with the transport device.

As guide for the guide wire or for a removal device for removal of the implant out of the packaging, the insertion aid can be provided in the passage of the front closure. The insertion aid comes out of the passage at its front, i.e. distal, end. The distal end preferably protrudes out of the packaging. By its rear, i.e. proximal end, the insertion aid ends on the implant or on the transport device, i.e. the catheter. The insertion device preferably comes out at a tip of the insertion catheter on which the implant is mounted. Alternatively the insertion aid can end inside the passage of the closure element so that the closure element can be compressed and sealed, as previously described, in the region without insertion aid. After the guide wire or the removal device have been inserted into the packaging, and preferably into the inner lumen of the catheter and/or of the stent, the insertion device can be removed, if necessary. For example the insertion aid can be provided as compressible tube or sleeve having an axially running parting line. The tube or the sleeve can then be torn open on the parting line and be taken off in radial direction by the guide wire. The insertion aid can also be formed by two half shells which are detachably connected together.

In a preferred application of the sleeve-like insertion element, this element is already disposed at least partially inside the closure element during the storage of the implant and of the catheter in the receptacle of the packaging. A distal catheter end, i.e. the catheter tip, comes out into the proximal end of the passage of the closure element, whereby a support wire protrudes outwardly, preferably already through the insertion aid. For example the insertion element is led into the closure element approximately up to the middle of the passage so that it covers over the passage distally halfway. The distal end of the insertion aid protrudes out of the packaging. The closure mechanism compresses the closure element so that the passage is halfway closed off proximally and also seals the catheter tip. In this state the implant and the catheter are enclosed inside the packaging and are protected against contamination. With a transfer of the implant the support wire is pulled out in the closed state of the closure element and the guide wire is pushed in. During pushing in of the wire the passage of the closure element is elastically widened.

Now the closure mechanism can be opened, so that the passage of the closure element opens and the insertion element can be inserted into the receptacle of the packaging. The distal end of the insertion element protruding out of the packaging can be coupled to a container or an insertion system. These can have, for example, the same closure mechanism as the packaging. Thus the distal end of the insertion element can be inserted in the closure mechanism of the container or of the insertion system and can be kept there. The implant, with the catheter as transport device, can now be transferred by the insertion element directly into the container or the insertion system without being exposed thereby to contamination.

The insertion element is preferably axially movable relative to the closure element and/or the packaging, or respectively the receptacle. The insertion element can thereby be positioned and aligned optimally relative to the catheter or stent.

It is advantageous to place the insertion element in the closure element in such a way that it covers over an inner side of the closure element, or respectively the seal. In this case the stent or the catheter with the stent can be removed from the packaging without coming into contact with the gasket. Thus the stent is prevented from being contaminated by sealant.

In another previously mentioned embodiment of the invention the insertion aid is formed by a sleeve-like insertion element which is disposed with an elastic area at least partially inside of the axial passage of the closure, or respectively of the closure element, and which has a distal end with an insertion opening for the removal device and/or a wire in the insertion element. Preferably the elastic region is formed on a proximal end of the substantially tubular, sleeve-like insertion element and comes out into the closure element or the receptacle.

The elastic region of the insertion element is compressible so that it abuts in a sealing way on the closure element and can be compressed with this element. The sleeve-like insertion element can be designed in an elastic way over its entire length. The insertion element can be adapted in diameter to the catheter by means of the closure mechanism. The distal end can be enlarged relative to the average diameter of the insertion element to facilitate the insertion of the wire. Preferably the distal end is also designed in an elastic way so that it can be widened or compressed during the coupling to the container or to an insertion system. An end region of the insertion element which comes to lie inside of the closure element can be designed elastic so that this end region can be compressed.

In another embodiment the insertion aid can also be provided by the tube element in which the implant is stored. In this case the insertion element extends through the packaging and the elastic region lies in a section that comes to lie inside of the closure element.

In an alternative embodiment the insertion aid can be integrated in the opening mechanism, or respectively in the closure of the packaging. For example the insertion element can be designed as one piece with the closure element.

It is also possible that the passage of the closure element, or respectively of the gasket, of the opening mechanism is adjustable in such a way that the passage forms an insertion aid with a first radius, and, with a second radius, which is greater than the first radius, is suitable for pushing through of the implant. The first radius is thereby large enough to be able to insert the insertion element or also the guide wire or a removal device into the receptacle of the packaging and to be able to position it relative to the implant, or respectively the transfer device, i.e. also to a catheter. After the positioning of the insertion element or of the guide wire, the radius of the passage can then be enlarged to the second radius. Then the implant can be pushed or also pulled through the passage with the second radius.

The insertion aid can be used advantageously independently of a coupling device and a transfer device. If, for example, a stent or catheter is stored in a conventional packaging and has to be removed from the packaging for use, the insertion element can be positioned manually on the end of the catheter and compressed. The wire can likewise be inserted manually into the distal end of the insertion element and be pushed through the insertion element into the catheter.

If the packaging or the packaging system according to the present invention is used with a transfer system for transferring an implant out of the packaging, the passage of the coupling device is coupled to the inlet port of the container or of the insertion system. An O-ring gasket can thereby be provided inside of the coupling device, which gasket comes to lie between the packaging and the container, or respectively the insertion system, and is adjacent thereto. The O-ring gasket can be clamped axially by the coupling mechanism so that it abuts tightly the packaging and container or respectively insertion system. Thus a penetration of contamination particles into the interior of the transfer system is prevented.

The coupling device can be formed by a sleeve-like insertion element as has been previously described as insertion aid. The opposite, distal end of the insertion aid is thereby coupled to the inlet opening of the container or of the insertion system. The coupling device preferably lies inside of the axial passage and inside of an axial passage of a container and/or of an insertion system. Thus the coupling is independent of the outer design of the packaging and of the insertion system.

In an embodiment the coupling device is coupled to a Y-connector or to a hemostasis valve of an insertion system as is normally used during the implantation of stents.

The packaging, the packaging system and the transfer system according to the invention facilitate the preparation of an implant for implantation, in particular of a stent, in that they simplify and accelerate the transfer of the implant out of the packaging into an insertion system. Moreover the risk of contamination of, or damage to, the implant is reduced during the transfer. In particular the implant can be kept at all times in a defined environment. Thus with use of a tubular or sleeve-like element as insertion aid the packaging system makes possible an uncomplicated, quick and contamination-free insertion of a wire or a removal device.

Preferred embodiments of the invention are presented in the following with reference to the drawings, which serve only as explanation and are not to be interpreted in a limiting way. Features of the invention disclosed from the drawings should be viewed individually and in any combination as belonging to the disclosure of the invention. In the drawings:

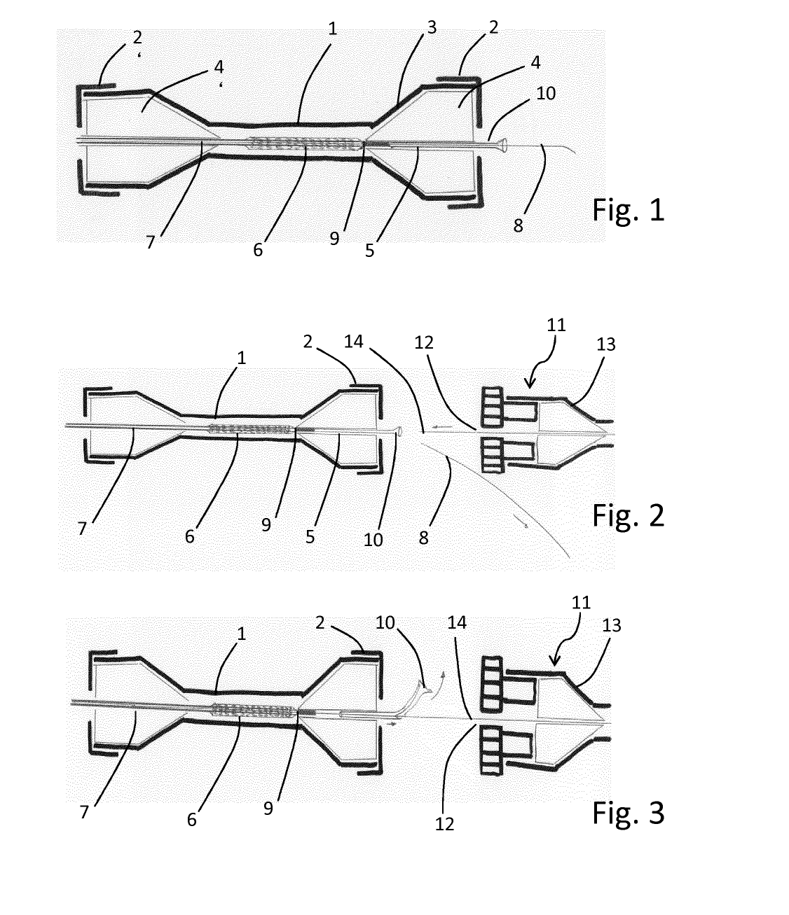

FIG. 1 shows a diagrammatical longitudinal section through a packaging for an implant in the form of a stent and with an insertion aid,

FIG. 2 shows a diagrammatical longitudinal section through a packaging with an insertion aid according to the present invention and an insertion system lying opposite,

FIG. 3 shows a diagrammatical longitudinal section according to FIG. 2 during removal of the insertion aid,

FIG. 4 shows a diagrammatical longitudinal section of a transfer system according to the present invention in a first embodiment,

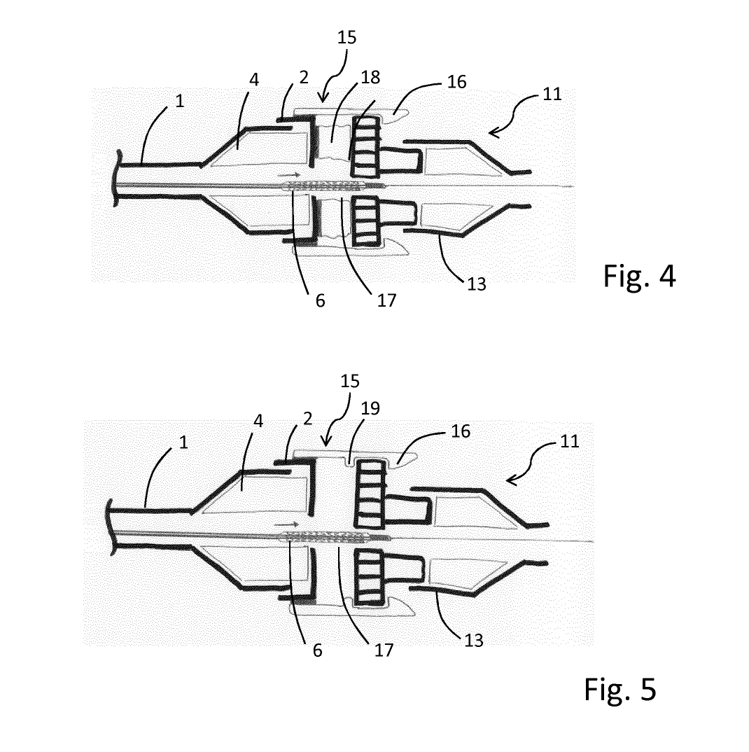

FIG. 5 shows a diagrammatical longitudinal section of a transfer system according to the present invention in a second embodiment,

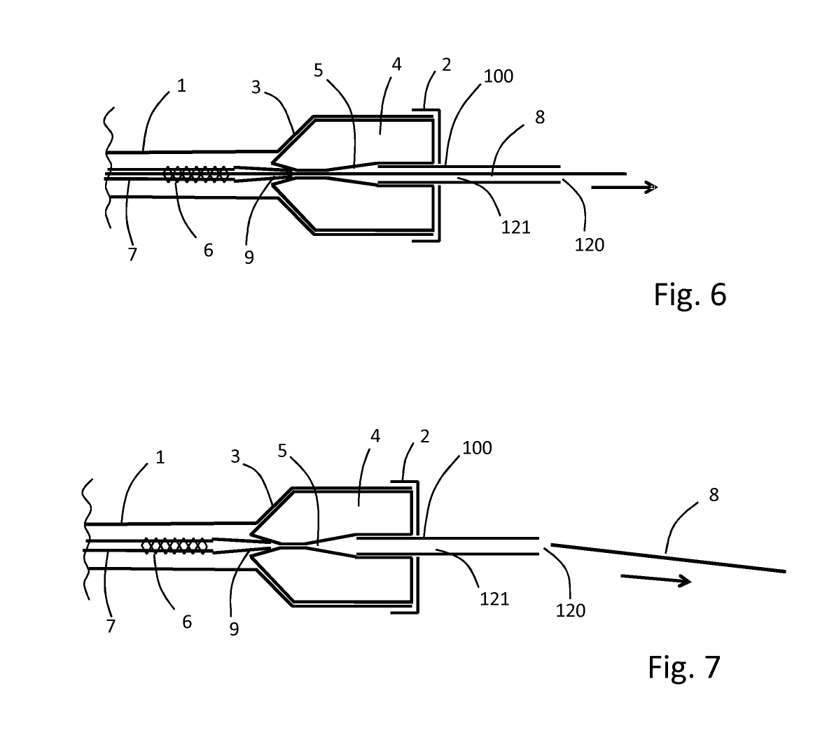

FIG. 6 shows a diagrammatical longitudinal section of a first embodiment example of a packaging system with a sleeve-like insertion element,

FIG. 7 shows a packaging system according to FIG. 6 with removed support wire,

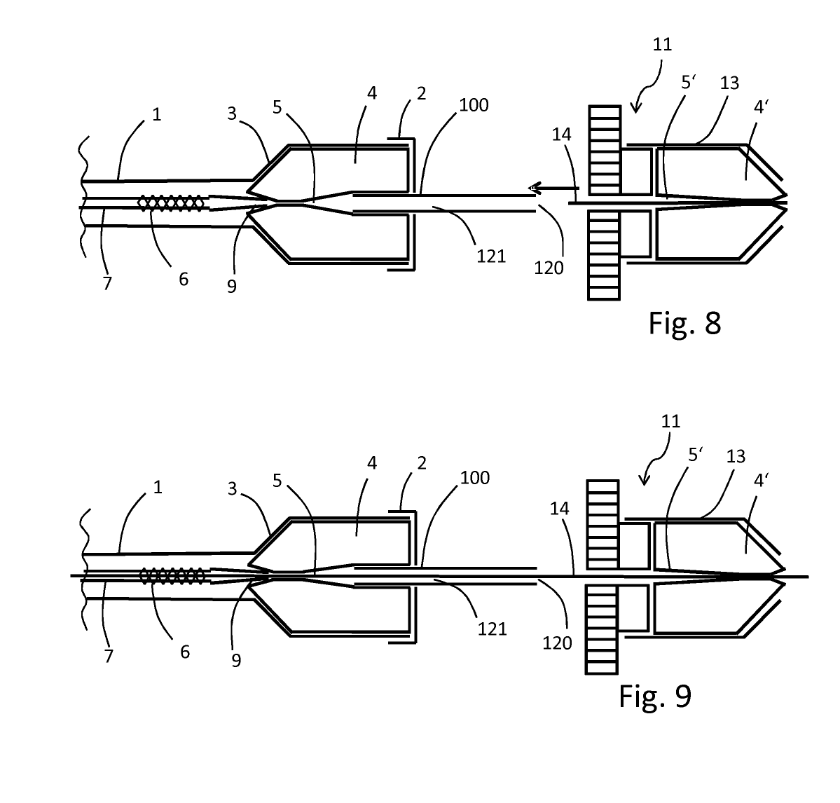

FIG. 8 shows a packaging system according to FIG. 6 for coupling to an insertion system,

FIG. 9 shows a packaging system according to FIG. 8 with inserted guide wire,

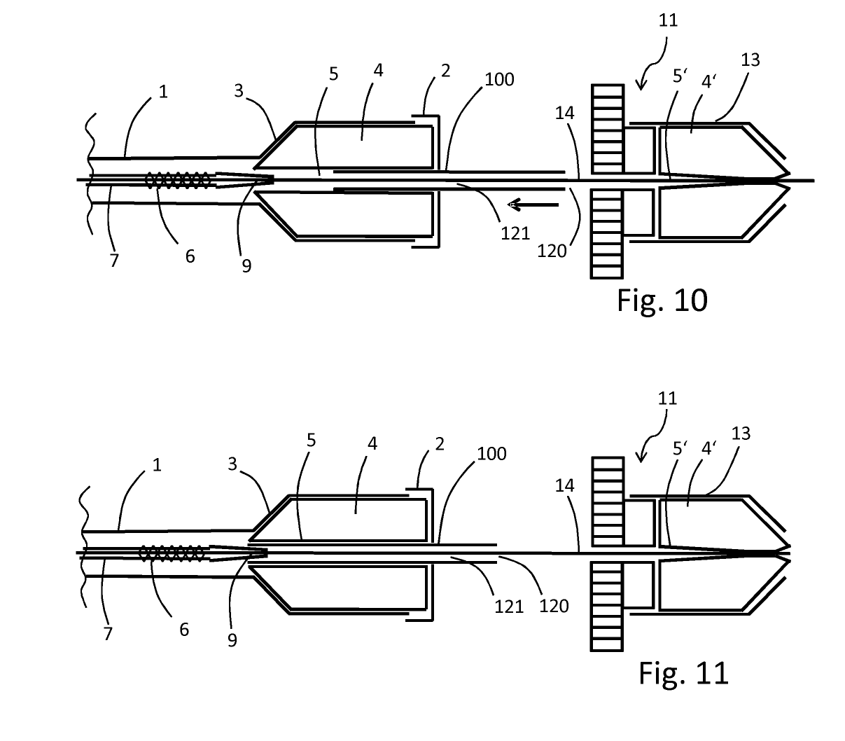

FIG. 10 shows a packaging system according to FIG. 9 with opened closure on the packaging,

FIG. 11 shows a packaging system according to FIG. 10 with further inserted insertion element,

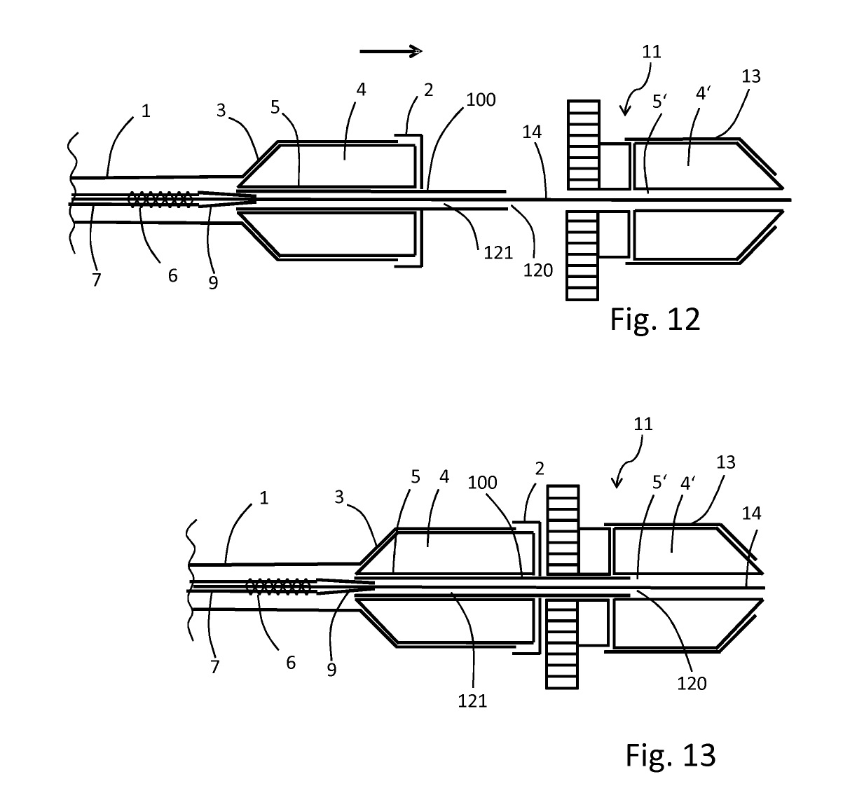

FIG. 12 shows a packaging system according to FIG. 11 with opened closure on the insertion system,

FIG. 13 shows a packaging system according to FIG. 12 with coupled insertion system,

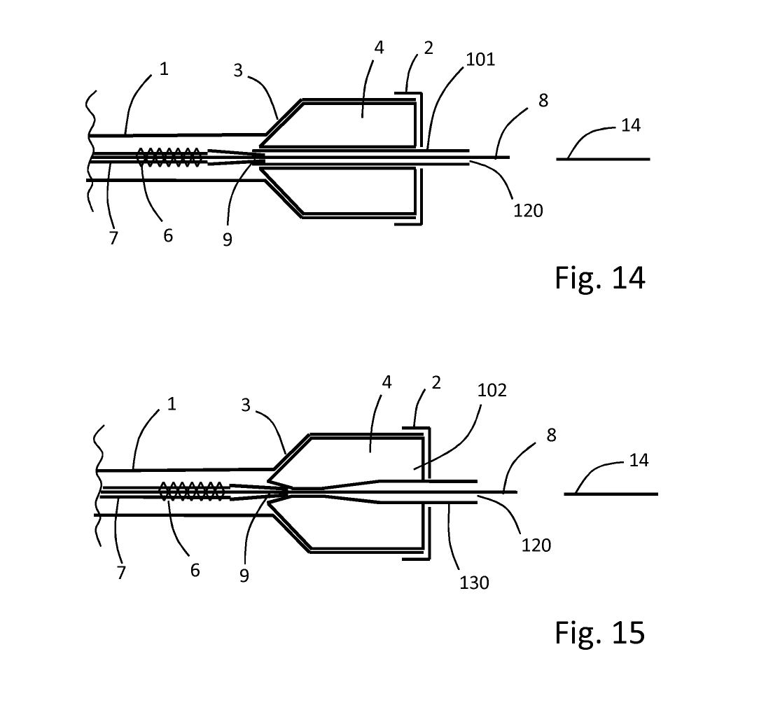

FIG. 14 shows a diagrammatical longitudinal section through a further embodiment example of a packaging system according to the invention with an insertion element with a flexible region,

FIG. 15 shows a diagrammatical longitudinal section through a further embodiment example of a packaging system according to the invention, in which the insertion element is provided by a closure element,

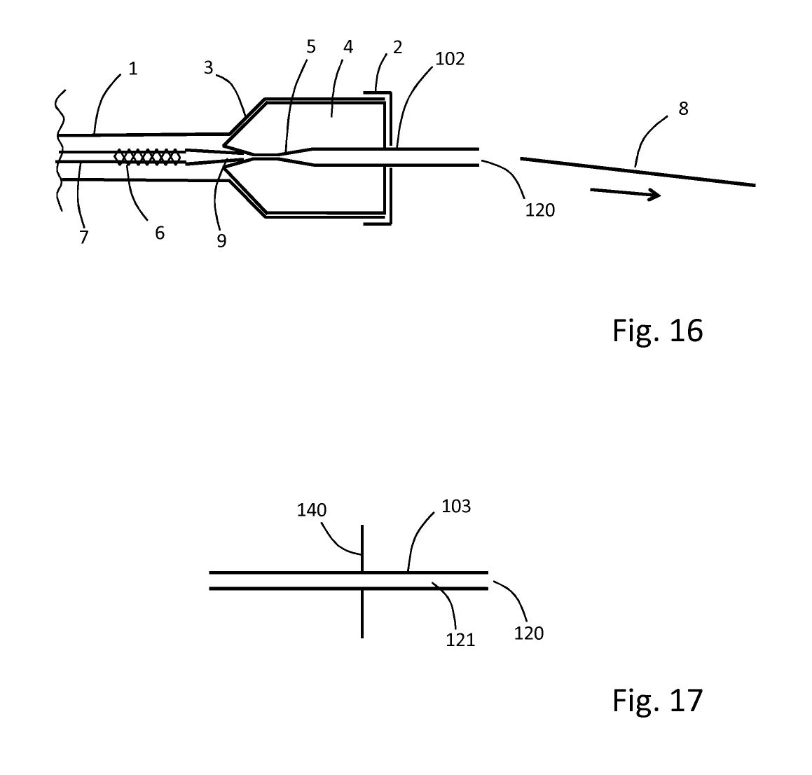

FIG. 16 shows a packaging system according to FIG. 15 with removed support wire,

FIG. 17 shows a schematic representation of a sleeve-like insertion element with positioning device,

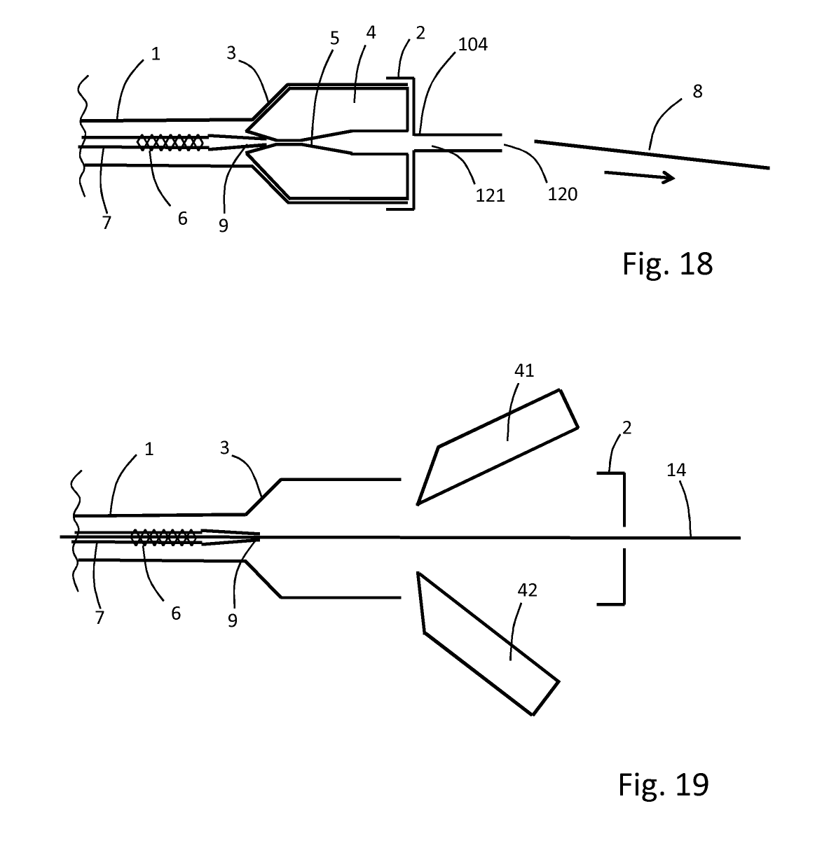

FIG. 18 shows a diagrammatical longitudinal section through a further embodiment example of a packaging system with an insertion element disposed on a closure of the packaging, and

FIG. 19 shows a diagrammatical longitudinal section through an embodiment example of a packaging system with a two-part closure element.

Shown in FIG. 1 is a packaging with a receptacle 1, which receives an implant and/or a device for use of the implant, as it finds application in the present invention. The packaging with the receptacle 1 is designed elongated and has at the front, distal end a first opening and at the opposite, rear, proximal end a second opening. The first and the second opening is closed in each case with a closure in the form of a cap 2 and 2′. A threaded connection is provided between the cap 2 and the receptacle along the circumference. The receptacle 1 is designed substantially cylindrical and has at the front and rear ends an expanded region with a conically running peripheral surface 3. Inserted in the expanded region in each case is an elastically deformable gasket 4 and 4′, which also serves as closure element and can thus form part of the closure. The gasket 4 has at its end protruding inwardly into the packaging 1 a conical peripheral surface which faces the conical peripheral surface 3 of the receptacle. A passage 5 extends centrally through the cap 2 and the gasket 4 from the inside of the receptacle 1 outwardly. By screwing the cap 2 on the receptacle 1 the gasket 4 is pressed against the peripheral surface 3, whereby it is displaced slightly axially and is radially compressed. In so doing the passage 5 in the gasket is closed so that the packaging is sealed. With unscrewing of the cap 2 from the receptacle 1 the gasket is released out of its elastic pretensioning and the passage 5 opens. This opening mechanism for opening and closing of the passage 5 is used both at the front as well as at the rear end of the packaging with the cap 2′ and the gasket 4′. The gasket, i.e. the closure element, can have a hardness between 30 and 70 Shore. A hardness of 45-55 Shore makes possible a reliable sealing of the packaging. An especially advantageous hardness is at 55 Shore.

Accommodated inside the packaging is an implant in the form of a stent 6. The stent 6 is stored in the packaging in an inert environment. The stent 6 is disposed on a dilation catheter 7, through which a support wire 8 runs. The stent 6 sits firmly on the dilation catheter 7, which is displaceable inside the packaging and therefore represents a transport device in the sense of the invention. The dilation catheter 7 and the support wire 8 extend through the passage 5 of the rear closure, namely through the cap 2′ and the gasket 4′. A tip 9 of the dilation catheter 7 comes out into an insertion aid in the form of a sleeve 10, which extends through the passage 5 of the gasket 4 and protrudes somewhat out of the cap 2. The protruding end is slightly expanded. The packaging is thus a component of a packaging system with an insertion aid for insertion of a removal device and/or a wire into an inner lumen of the stent 6 and/or of the catheter 7 according to the invention.

Shown in the FIGS. 2 to 5 is the packaging from FIG. 1 as part of a transfer system for removal of the implant out of the packaging and for insertion of the implant into an insertion system 11. In the embodiment example shown the insertion system 11 has a hemostasis valve 13 with a sealing mechanism and comprises an inlet port 12, which is provided on the hemostasis valve 13. The sealing mechanism comprises a turn-lock closure with a conical surface and a gasket such that through rotation of the turn-lock closure in the form of a cap the gasket is pressed against the conical surface and thus seals. The inlet port 12 of the insertion system 11 comes out of the hemostasis valve 13, or respectively the turn-lock cap, of the insertion system 11. Inside the insertion system 11 runs a guide wire 14, which protrudes out of the inlet port 12. The guide wire 14 serves in the usual way to guide the stent 6 with the dilation catheter 7 into a body lumen in which the stent 6 is supposed to be placed.

As shown in FIG. 2, as preparation for a transfer of the stent 6 into the insertion system 11, the support wire 8 is pulled out of the receptacle 1, or respectively out of the dilation catheter 7. Then the guide wire 14 is inserted by means of the insertion aid 10 into the dilation catheter 7. As shown in FIG. 3, the insertion aid 10 can be taken off after the insertion of the guide wire 14. For this purpose the sleeve can be torn off in longitudinal direction, e.g. along a parting line. Furthermore the closure 2, or respectively the passage 5 is opened to a second radius to the extent that the stent is able to be pushed through by means of the dilation catheter 7. However, in principle the closure 2, or respectively the passage 5, can be completely opened only after the coupling.

Shown in FIG. 4 is a first embodiment of a coupling device 15 according to the invention. The coupling device 15 is designed as separate coupling piece which is inserted between the packaging 1 and the insertion system 11. The coupling device 15 is attached on the outer circumference of the cap 2 of the packaging 1 by means of a screw connection. The coupling device 15 has snap arms 16 with hooks. For fastening of the insertion system 11, or respectively its hemostasis valve 13, the snap arms engage behind an axial stop on the hemostasis valve 13. An O-ring gasket 18 is borne in the coupling device between the packaging 1 and the front end of the hemostasis valve 13, which gasket firmly abuts in engaged state of the snap arms and is at least slightly compressed. The thereby arising counterforce brings about stability of the engaged snap arms 16 and thus the snap connection. A passage 17 extends through the coupling device 15, i.e. through the O-ring gasket 18, which passage comes out into the passage 5 and also at the inlet port 12. The stent can now be pushed, mounted on the dilation catheter, through the passage 5 in the gasket 4, the passage 17 in the coupling device 15 and the inlet port 12 of the hemostasis valve into the insertion system 11. The stent is thereby not exposed at any time to a contaminating environment and is protected against manual grasp.

Shown in FIG. 5 is another embodiment of a coupling device 15 according to the invention. Compared with FIG. 4, this differs in that no O-ring gasket 18 is provided. Instead inwardly protruding projections 19 are disposed on the snap arms, which projections serve as stop for the front side of the inlet port 12, for example of the turn-lock cap of the hemostasis valve 13, while the hooks of the snap arms engage. The snap arms in this embodiment are firmly attached on the cap 2, so that the coupling device 15 here is not releasable like that from FIG. 4. Moreover in this embodiment the insertion aid is provided in that the passage of the gasket 4 is expandable initially to a first diameter which is just sufficient in order to be able to guide the guide wire to the dilation catheter 7, whereby the guide wire is positioned on an inlet of the dilation catheter. Then the passage of the gasket 4 is expandable to a second diameter, which is greater than the first, so that the stent with the dilation catheter 7 is able to be pushed through.

FIGS. 6 to 13 show a further example of a packaging system according to the present invention. Components of the packaging system which correspond to the components of the previously described system are designated by the same reference symbols and reference is made to the previous description.

The packaging system comprises a packaging with a receptacle 1 for the implant in the form of a stent 6 and a catheter in the form of the dilation catheter 7, whereby the receptacle 1 has at its distal end a closure 2 and an opening or respectively actuation mechanism with a closable and expandable axial passage 5. A sleeve-like insertion element 100 with a continuous channel 121 is disposed partially in the axial passage 5 of a closure element in the form of a gasket 4, as previously described, so that a proximal end of the insertion element 100 comes to lie inside the gasket 4. The channel 121 corresponds to the passage 17 from the above-described example. An opposite distal end forms an insertion opening 120 for insertion of a removal device and/or a wire, in this example for a guide wire 14, into the insertion element 100. The closure is formed by the cap 2 <and> the closure element. The sleeve-like insertion element 100 is in this example an almost rigid element that is not supposed to be compressed by the closure mechanism. The material is therefore selected to be harder than that of the closure element. The insertion element can however be designed to be slightly deformable and/or flexible. Essential is that the channel 121 remains open despite a compression of the closure element.

Provided that the catheter 7 has a support wire 8, the insertion element 100 is disposed over the support wire 8 and is inserted along this wire into the passage 5. The threading of the insertion element on the support wire takes place outside of the packaging. The support wire thus serves as guide for the insertion element 100.

As shown FIG. 6, the insertion element 100 is pushed into the passage to approximately half the length of the passage 5 in the closure element and covers the inner circumference of the closure element. The insertion element can also be inserted more or less to half the length. Essential is that the non-covered proximal region of the closure element is able to be compressed by the previously described closure mechanism by means of the cap 2. The catheter 7 is thereby disposed inside the passage, preferably with the tip at the proximal end of the passage 5. Through the compression of the gasket 4 the insertion element 100 is fixed in the distal region of the gasket, and the proximal region is closed in such a way that it seals off the catheter 7 at its tip 9. At the same time the gasket 4 seals at the conical peripheral surface. The receptacle 1 with the stent is thus sealed all around, and the stent is protected against contamination.

Then the support wire 8, if present, is removed out of the catheter 7, as shown in FIG. 7. The opening mechanism or respectively actuation mechanism does not have to be opened for this purpose. The insertion element 100 and the closure element, i.e. the gasket 4, surround the support wire elastically, so that it is possible to pull the latter against a minimal frictional force, which is caused by the elastic material, through the insertion and closure element. The sealing effect of the gasket 4 is thereby preserved.

Shown in FIGS. 8 and 9 is how the guide wire 14 of an insertion system 11 is inserted at the distal end of the insertion element 100 into its insertion opening 120. For this purpose the guide wire 14 is extended out of the insertion system 11 and is pushed through the insertion opening 120 into the insertion element 100. The guide wire 14 is pushed completely through the channel 121 of the insertion element 100 and further through the compressed region of the gasket 4 into the catheter 7, as can be seen in FIG. 9. Already in this state the closure mechanism can be opened, whereby the gasket 4 expands and the implant can be removed with a removal device along the guide wire 14 or can be pushed out with the transport device.

Preferably the sleeve-like insertion element 100 is used, according to the invention, as coupling device for coupling the packaging to the insertion system 11 or a container, as shown in FIGS. 8-13. For this purpose the insertion element 100 is coupled, by the distal end, to the inlet port 12 of a container or of an insertion system 11 for insertion of the stent into a body lumen.

In a preferred embodiment the insertion system 11 has a hemostasis valve 13 with a sealing mechanism, and the guide wire 14 comes out of the insertion system 11 and the hemostasis valve 13, as can be seen from FIGS. 8 to 13. In this case the distal end of the insertion element 100 with the insertion opening 120 can be inserted into the sealing mechanism of the insertion system 11. Preferably the sealing mechanism closes off the outer circumference of the insertion element 100 in a tightly sealing way. The stent 6 and the dilation catheter 7 can be led from the protected environment in the receptacle 1 of the packaging directly into the insertion element 100, through this element directly into the protected environment of the insertion system 11 and to a body lumen without being exposed to a contaminating environment and without coming into contact with possibly contaminating sealing elements of the packaging or of the insertion system 11.

Shown in FIG. 10 is the closure mechanism of the packaging in an opened state so that the insertion element 100 is movable axially and rotationally inside the passage 5. The insertion element 100 is pushed further along the guide wire 14 into the packaging until the proximal end of the channel 121 encloses the catheter tip 9, as shown in FIG. 11. The insertion element 100 can be firmly held, e.g. by means of the sealing mechanism, if necessary. Then the sealing mechanism of the hemostasis valve 13 is opened (FIG. 12), and the packaging can be moved until the stop on the hemostasis valve (FIG. 13). The insertion element 100 is thereby pushed into a passage 5′ of the hemostasis valve and can, if necessary, be firmly held, e.g. by means of the sealing mechanism. Now the dilation catheter 7, with the stent 6 mounted thereon, can be pushed forward through the insertion element 100 into the insertion system 11 and led along the guide wire 14 to the site of application.

Shown in FIG. 14 is still another example of a packaging system according to the present invention. In it the packaging system comprises an insertion aid, which is provided by a sleeve-like insertion element 101, which is designed elastic at least in one region on the proximal end. The proximal end is disposed at least partially inside the axial passage 5 of the closure. During use of the elastic insertion element 101 as insertion aid, the elastic region of the insertion element 101 is positioned and compressed inside the receptacle 1 on a catheter end, i.e. the distal catheter tip 9, such that it is adjacent in a flush way to the catheter end. In the present example the proximal end of the insertion element 101 overlaps the catheter end so that the catheter end comes to lie inside the insertion element 101 or respectively the catheter end abuts on the insertion element. For this purpose the opening mechanism is opened, so that the closure element, or respectively the gasket 4, and thereby also the passage 5 are in an opened state and the insertion element 101 can be pushed through the closure element. The insertion element 101 is axially movable relative to the closure and to the packaging along an axis of the packaging or respectively the axial passage 5. For positioning of insertion element 101 and catheter 7, the catheter in the sense of the above-described transport device can also be pushed in direction of the insertion element 101.

The insertion element 101 is preferably pushed through the entire passage 5 until the proximal opening of the insertion element 101 comes out in the region of the receptacle, in which the stent 6 and the catheter 7 are stored. In so doing the elastic region at the proximal end of the insertion element 101 should come to lie inside the closure element, or respectively the gasket 4. The elastic end can thereby be compressed and closed by means of the actuation mechanism.

The closure element and the insertion element can thereby come to lie on one another in a sealing way. The insertion element 101 can also be designed completely elastic and be compressed over its length which extends within the closure element.

Now the guide wire 14 at the distal end of the insertion element 101 can be inserted into its insertion opening 120 and be pushed through the elastically designed, proximal end into the catheter. The opening mechanism or respectively actuation mechanism thereby remains closed so that the proximal end of the insertion element 101 is adjacent to the catheter end in a flush way. Thus the proximal opening of the insertion element 101 is connected to the opening of the catheter 7, and the guide wire 14 is led from the insertion element 101 through the catheter opening into the inner lumen of the catheter and of the stent. The insertion element 101 and the closure element, or respectively the gasket 4, are in fact in a compressed, closed state so that the passage 5 is closed. The proximal end of the guide wire can however deform the elastic material of insertion element 101 and gasket 4 in such a way that the guide wire can be pushed through the passage 5. In so doing the insertion element 101 covers the inside of the closure element, or respectively of the gasket 4, over its length so that the guide wire is not able to be contaminated by the gasket or sealant located on the gasket. At the same time the closure element abuts in a sealing way the insertion element 101 and the conical peripheral surface 3 of the packaging, so that once again no contamination of the interior of the receptacle 1 can take place.

The insertion element 101 can also serve as coupling device for coupling to a container or an insertion system 11, as is foreseen with a packaging according to the invention. For this purpose the distal end of the insertion element 101 can also be designed elastic and can be coupled to an inlet port 12 of a container or of an insertion system 11 for insertion of the stent into a body lumen. For example, by means of frictional connection, the elastic end can be pushed over a connection piece having an inlet opening or, like the insertion element 100, it can be disposed in the passage 5′ of the insertion system.

In another variant of a packaging system according to FIGS. 15 and 16 an insertion element 102 is designed as one piece with a closure element, in particular with the gasket 4, of the closure 2. In this case the passage 5 assumes the function of the channel 121 of the previous embodiment examples.

The closure element with its elongated form and the axial passage 5 thus form a sleeve-like element. A threading of the insertion element 102 on a support wire and an insertion of the closure element are not necessary in this embodiment example. For insertion of a wire, such as e.g. the aforementioned guide wire 14, the catheter 7 is pushed until it abuts on the closure element. The closure element has a distal extension 130 for insertion of the wire 14, which has the insertion opening 120 and runs through the passage 5. The extension 130 preferably protrudes axially out of the closure through the cap 2. During compression of the closure element by means of the actuation mechanism the extension 130 remains at least almost uncompressed, so that the insertion opening 120 remains open also in closed state. Further use takes place in a way similar to the previous examples. The support wire 8 is removed, as shown in FIG. 16, and a guide wire 14 is inserted. In particular the extension 130 can also be coupled to an insertion system 11 or a container.

Shown in FIG. 17 is a variant of an insertion element 103, which has a positioning device 140 for positioning of the insertion element 103 inside the packaging or respectively of the closure element. The positioning device 140 can be formed e.g. by a stop protruding radially from the outer circumference of the sleeve-like insertion element 103. The stop can be disposed e.g. between the cap 2 and the gasket 4. The insertion element 103 is thus secured against an axial movement. Thus it can be prevented that the insertion element 103 is pushed along during forward pushing of the catheter 7. It is also ensured that the insertion element 103 is not pushed along into the insertion system 11. In principle an axial displacement of the insertion element 103 can also be counteracted through a frictional connection with the gasket.

Presented in FIG. 18 is a variant of an insertion element 104, which is firmly mounted on the cap 2. The channel 121 forms an axial prolongation of the passage 5 of the gasket 4. The gasket 4 is designed in such a way that it is compressible only in a proximal region by means of the closure mechanism. A distal region, in particular the distal opening of the passage 5 is not compressed or is only minimally compressed. This can be achieved e.g. through different materials in the distal and proximal region or through a suitable length of the gasket 4. With sufficient length the distal region is not compressed by the conical surface 3. In this variant of the insertion element 104 a guide wire is led directly into the passage 5 of the gasket 4. Since, in the proximal region, the gasket 4 encloses the tip 9 of the catheter 7, the wire is led further into the catheter.

In the embodiment examples shown, the distal end of the insertion element can protrude out of the packaging and can be enlarged relative to the average diameter of the insertion element. The insertion of the wire 14 is thereby facilitated.

Embodiment examples for a packaging, a packaging system and a transfer system according to the invention with a stent and a dilation catheter were shown in the figures. In principle the packaging, the packaging system and the transfer system are however also suitable for other types of implants that are supposed to be transferred out of a packaging into another container or an insertion system, i.e. from a protected environment into another protected environment or transferred for a utilization. Furthermore the features of the examples shown can be combined with one another advantageously by one skilled in the art. Therefore the right remains reserved to claim patent protection also for a combination of features that is not shown in a single example but is covered by the inventive concept.

Shown in FIG. 19 is a closure mechanism with axially divided closure element. The closure element has two halves 41 and 42, which each form a half shell with an axially running depression. In the state of being placed on one another the depressions form together the passage 5 of the closure element. After use as seal, the two-part closure can be pulled out of the packaging and taken apart so that it can be removed by a guide wire running in the packaging. The two-part closure element can also be used as insertion aid, as previously described.

### LIST OF REFERENCE NUMERALS

1 packaging/receptacle
2, 2′ cap
3 conical peripheral surface
4, 4′ gasket
5, 5′ passage
6 stent
7 dilation catheter
8 support wire
9 tip
10 insertion aid
11 insertion system
12 inlet port
13 hemostasis valve
14 guide wire
15 coupling device
16 snap arms
17 passage
18 O-ring gasket
19 projections
41 closure element half
42 closure element half
100, 101, 102, 103, 104 insertion element
120 insertion opening
121 channel
130 extension
140 positioning device

1. Packaging for an implant or a device for use of an implant, which comprises a receptacle for the implant or a device for use of an implant, wherein the packaging comprises on a front end a closure with an opening mechanism and on a rear end a transport device for moving the implant, or the device for use of the implant, relative to the packaging through the closure, wherein adjacent to the closure there being a coupling device with a passage, which coupling device is movable relative to the receptacle, which passage is connectible to an inlet port of a container or an insertion system.

2. Packaging according to claim 1, wherein the coupling device is a separate coupling piece or is provided in a fixed way on the packaging.

3. (canceled)

4. Packaging according to claim 1, wherein the opening mechanism of the closure has an expandable axial passage.

5. Packaging according to claim 1, wherein the closure has an elastically deformable gasket including the axial passage which, by means of a rotation element, is deformable such that the passage opens or closes.

6. Packaging according to claim 1, wherein the passage of the coupling device extends in axial direction and in coupled state connects to the axial passage of the closure.

7-8. (canceled)

9. Packaging according to claim 1, wherein provided in the passage of the front closure is a removable insertion aid, which, at its front end, comes out of the passage and at its rear end terminates at the implant or at the device for use of an implant.

10. (canceled)

11. Packaging according to claim 5, wherein the passage of the gasket of the opening mechanism is adjustable in such a way that, with a first radius, the passage forms an insertion aid and, with a second radius, which is greater than the first radius, is suitable for pushing through the implant.

12. (canceled)

13. Transfer system according to claim 15, wherein inside the coupling device an O-ring gasket is provided which lies adjacent to the front end of the packaging and to the container, or respectively to the insertion system.

14. (canceled)

15. Packaging system with a packaging for an implant and/or a catheter and with an insertion aid for insertion of a removal device and/or a wire into an inner lumen of the implant and/or of the catheter in the packaging, comprising:
the packaging according to claim 1, wherein said a receptacle is configured for the implant and/or the catheter, and has at a distal end a closure and an opening mechanism with a closable and expandable axial passage, and
the insertion aid is provided by a sleeve-like insertion element which is disposed at least partially within the axial passage of the closure, or which is formed by the closure element, wherein a distal opening of the insertion element forms an insertion opening for the removal device and/or the wire into the insertion element.

16. Packaging system according to claim 15 wherein the insertion element is axially moveable relative to the closure and/or the receptacle.

17. (canceled)

18. Packaging system according to claim 16, wherein a proximal opening of the insertion element is disposed in such a way that it encloses a tip of the catheter or of the implant in the packaging.

19. Packaging system according to claim 15, wherein the distal end of the insertion element is enlarged relative to the average diameter of the insertion element.

20-33. (canceled)

34. Packaging system according to claim 15, wherein the insertion element forms a coupling device for coupling to a container or an insertion system.

35. Packaging system according to claim 34, wherein the coupling device is situated inside the axial passage, and inside an axial passage of a container and/or of an insertion system.

36. Packaging system according to claim 15, wherein the sleeve-like insertion element comprises at least one elastic region which is supported inside the axial passage.

37. Packaging system according to claim 36, wherein the at least one elastic region of the insertion element is compressible in a sealing way.

38. Packaging system according to claim 15, wherein the insertion element is completely elastic.

39. (canceled)
